(19) United States
(12) Patent Application Publication
(10) Pub. No.: US 2025/0278659 A1
(43) Pub. Date: Sep. 4, 2025

(54) SYSTEM FOR CALCULATING QUANTUM CHEMISTRY BASED ON QUDIT

(71) Applicant: KOREA INSTITUTE OF SCIENCE AND TECHNOLOGY, Seoul (KR)

(72) Inventors: Hyang-Tag LIM, Seoul (KR); Kangmin HU, Seoul (KR); Yong-Su KIM, Seoul (KR); Seung-Woo LEE, Seoul (KR)

(73) Assignee: KOREA INSTITUTE OF SCIENCE AND TECHNOLOGY, Seoul (KR)

(21) Appl. No.: 18/667,176

(22) Filed: May 17, 2024

(30) Foreign Application Priority Data
Feb. 29, 2024 (KR) 10-2024-0029939

(51) Int. Cl. G06N 10/60; G16C 10/00

(52) U.S. Cl. CPC G06N 10/60; G16C 10/00

(57) ABSTRACT

Disclosed is a system for calculating quantum chemistry based on a qudit according to one embodiment, the system comprises the quantum processing unit configured to including a first updating unit configured to update a diffraction image to a t-th diffraction image on the basis of a t-th parameter obtained from a classical processing unit; and a second updating unit configured to allow a photon in a 0-th state input to the quantum processing unit to enter the t-th diffraction image update to a photon in a t-th state, and classical processing unit configured to calculate expectation values of the first to t-th Hamiltonians corresponding to photons in first to t-th state, and determine the t-th parameter on the basis of an expectation value of the Hamiltonian of a (t−1)-th parameter.

### CROSS REFERENCE TO RELATED APPLICATION

This application claims the benefit under 35 USC 119 (a) of Korean Patent Application No. 10-2024-0029939 filed on Feb. 29, 2024, in the Korean Intellectual Property Office, the entire disclosure of which is incorporated herein by reference for all purposes.

### BACKGROUND OF THE INVENTION

### Field of the Invention

Disclosed embodiments relate to a technique for calculating a quantum chemical problem based on a qudit, and more specifically, a technique for solving a quantum chemical problem by adjusting parameters for a diffraction image.

### [Description of Government-Sponsored Research]

This research was supported by the Ministry of Science and ICT [Project Number: 1711181243, Subproject Number: 2022M3E4A1043330, Project Title: Quantum Computing Technology Development Project, Project Title: Research on High Brightness and High Quality Quantum Entanglement States Generation Using Noncritical Phase Matching].

This research was supported by the Ministry of Science and ICT [Project Number: 1711195718, Subproject Number: 00222863, Project Title: Leading Quantum Technology Development (Quantum Sensor), Project Title: Development of multiparametric quantum light-based sensing technology capable of overcoming decoherence and light loss].

### Description of the Related Art

Quantum chemistry calculation is a technique that identifies the properties of new materials and may be used in the development of new medicines, in the battery industry, etc. and is studied in various systems including optics, superconductivity, ion traps, etc. However, the quantum chemistry calculation usually includes complex problems and requires significant resources to solve the problems

In particular, a higher dimensional Hilbert space is needed to improve the precision of calculating quantum chemistry. In this case, the higher dimensional Hilbert space requires a higher number of qubits.

However, there is a limitation to an increase in dimensionality. This is because calculations between the higher number of qubits require more quantum gates, but an infinite expansion in dimensionality is inappropriate since the increased number of gates leads to a decrease in fidelity.

However, since a single-qudit based quantum chemistry calculation may be solved using a single gate, the limitation on expansion in dimensionality are removed. Moreover, when a photon is given with an orbital angular momentum state, having a qudit state, dimensional extensibility may be ensured.

### Documents of Related Art

Korean Patent Application Laid-Open No. 10-2021-0138713 (published on Nov. 19, 2021)

### SUMMARY OF THE INVENTION

Disclosed embodiments are aimed at repeatedly assigning an orbital angular momentum state to a single photon to solve the best solution of a quantum chemical problem.

According to one embodiment, there is provided a system for calculating quantum chemistry based on a qudit, the system comprises: a quantum processing unit configured to include one or more optical components; and a classical processing unit configured to include one or more processors and memory, in which the quantum processing unit (hereinafter, N is a natural number equal to or greater than one, and t is a natural number equal to or greater than one and equal to or less than N) is configured to including: a first updating unit, including one or more optical components, configured to update a diffraction image to a t-th diffraction image on the basis of a t-th parameter obtained from the classical processing unit; and a second updating unit, including one or more optical components, configured to allow a photon in a 0-th state input to the quantum processing unit to enter the t-th diffraction image update to a photon in a t-th state, in which a first update unit and a second update unit are alternately updated N number of times sequentially from a first time to a N-th time, respectively, and in which the classical processing unit is configured to: calculate expectation values of the first to t-th Hamiltonians corresponding to photons in first to t-th state, and determine the t-th parameter on the basis of an expectation value of the Hamiltonian of a (t−1)-th parameter. In this case, a 0-th parameter is determined on the basis of an arbitrary value.

The classical processing unit may be further configured to calculate an eigenvalue of the Hamiltonian corresponding to a photon in an N-th state to further calculate a ground state of the photon.

The classical processing unit may be configured to determine a value of the t-th parameter such that an expectation value of the Hamiltonian of the t-th parameter is less than the expectation value of the Hamiltonian of the (t−1)-th parameter.

The classical processing unit may be configured to determine a minimum value of the t-th parameter that causes an expectation value of the Hamiltonian of the t-th parameter to be less than the expectation value of the Hamiltonian of the (t−1)-th parameter.

The classical processing unit may be configured to determine a value of the t-th parameter when the difference between a value of the (t−1)-th parameter and the value of the t-th parameter is greater than or equal to a preset tolerance.

The photon may be a single one.

The system may further include a photon generating device configured to include one or more optical components, in which the photon generating device may be configured to generate a single photon on the basis of at least one of spontaneous parametric down conversion, a point defect, or a quantum dot. a photon generating device,

The photon generating device may be configured to generate a pair of photons on the basis of the spontaneous parametric down conversion, and detect an other photon when a single photon separated from the pair of photons is input to the quantum processing unit.

The quantum processing unit may further include a measuring unit, including one or more optical components, configured to measure an expectation value of a Pauli operator corresponding to the photon in the t-th state, and in which the classical processing unit may be configured to calculate an expectation value of the Hamiltonian of the photon in the t-th state on the basis of the expectation value of the Pauli operator corresponding to the photon in the t-th state.

The first updating unit may be configured to generate a diffraction image by a first spatial light modulator provided in the first update unit.

The measuring unit may be configured to measure an expectation value of a Pauli operator corresponding to a photon in the t-th state by a second spatial light modulator provided in the measuring unit.

The second updating unit is configured to allow the photon to enter the diffraction image and update to have a qudit state in which the photon possesses an orbital angular momentum.

Disclosed embodiments update an orbital angular momentum state on a single photon to solve for the optimal Hamiltonian expectation value, instead of the multiple qubit-based gate calculations used in conventional techniques, which may reduce the use of resources required for the quantum chemistry calculation.

Disclosed embodiments give an orbital angular momentum state to a single photon, ensuring a high-dimensional space of multiple orthogonal bases, which may resolve the problem of dimensional extensibility that has been limited in the conventional quantum chemistry calculation.

Disclosed embodiments restrict updating of the parameters that generate the orbital angular momentum state within a tolerance, which may provide efficient parameter optimization by avoiding unnecessary time and calculations for convergence.

### BRIEF DESCRIPTION OF THE DRAWINGS

FIG. 1 is a view for describing a system for calculating quantum chemistry based on a qudit, according to one embodiment.

FIG. 2 is a view for describing a system for calculating quantum chemistry based on a qudit, according to an additional embodiment.

FIG. 3 is a flowchart for describing an operation sequence of one example performed by the system for calculating quantum chemistry based on a qudit according to one embodiment.

FIG. 4 is a flowchart for describing an operation sequence of another example performed by the system for calculating quantum chemistry based on a qudit according to one embodiment.

FIG. 5 is a flowchart for describing an operation sequence of one example performed by the system for calculating quantum chemistry based on a qudit according to the additional embodiment.

FIG. 6 is an exemplified view for describing a state of a photon being changed by the system for calculating quantum chemistry based on a qudit, according to one embodiment.

FIG. 7A and FIG. 7B are result graphs for describing a calculation efficiency of the system for calculating quantum chemistry based on a qudit according to one embodiment.

FIG. 8 is a result graph for describing a calculation precision of the system for calculating quantum chemistry based on a qudit according to one embodiment.

### DETAILED DESCRIPTION OF THE INVENTION

Hereinafter, specific exemplary embodiments of one embodiment will be described with reference to the drawings. The following detailed description is provided to assist in the comprehensive understanding of a method, an apparatus, and/or a system described in the present specification. However, the exemplary embodiments are provided only for illustrative purpose, and the present invention is not limited thereto.

In addition, in the description of the exemplary embodiments, the specific descriptions of publicly known technologies related with the present invention will be omitted when it is determined that the specific descriptions may unnecessarily obscure the subject matter of the exemplary embodiments. In addition, the terms used herein are defined considering the functions in the present invention and may vary depending on the intention or usual practice of a user or an operator. Therefore, the definition of the present invention should be made based on the entire contents of the present specification. The terms used in the detailed description are provided only for describing the exemplary embodiments and should not be restrictive. Unless explicitly used otherwise, singular expressions include plural expressions thereof. In the present specification, the terms “comprises,” “comprising,” “includes,” “including,” “containing,” “has,” “having” or other variations thereof are provided to indicate specific components, numbers, steps, operations, component, elements, and some or combinations thereof, and it should not be construed to exclude the presence or possibility of one or more other components, numbers, steps, operations, component, elements, and some or combinations thereof other than those disclosed.

Terms “first”, “second”, and the like may be used to describe various constituent elements, but the constituent elements are of course not limited by these terms. These terms are merely used to distinguish one constituent element from another constituent element. Therefore, the first constituent element mentioned hereinafter may be the second constituent element within the technical spirit of the present invention.

In addition, the embodiments disclosed in the present specification may have a configuration that is hardware as a whole, hardware partially, software partially, or software as a whole.

In the present specification, “unit,” “device,” and the like refer to any combination of hardware, software, and the like. For example, the unit, device, and the like may refer to the integrated photonic circuit itself or to software connected to the hardware to drive the integrated photonic circuit.

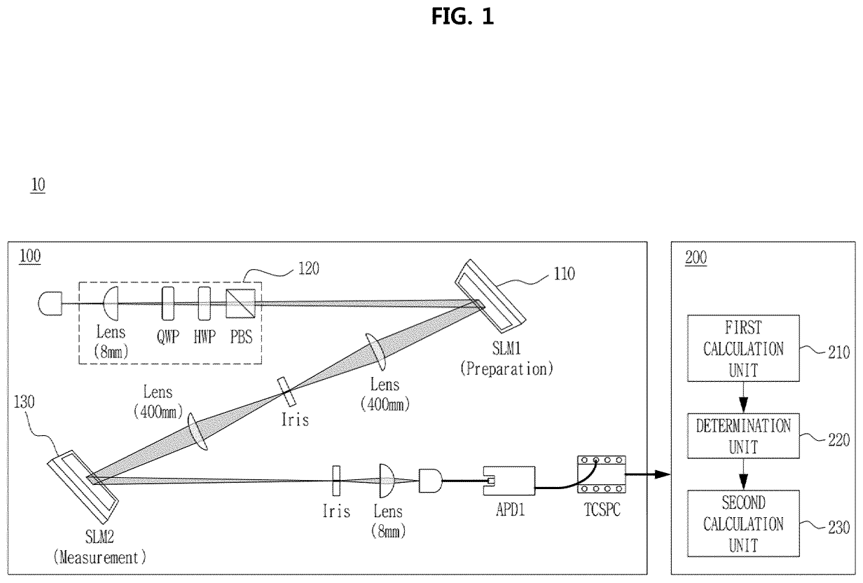

FIG. 1 is an exemplified view for describing a system 10 for calculating quantum chemistry based on a qudit, according to one embodiment.

With reference to FIG. 1, the system 10 for calculating quantum chemistry based on a qudit according to one embodiment includes a quantum processing unit (QPU) 100 and a classical processing unit (CPU) 200.

The system 10 for calculating quantum chemistry based on a qudit is a system used to solve quantum chemistry problems in qudit units. Here, a qudit means a quantum state in d-dimension. That is, the system 10 for calculating quantum chemistry based on a qudit may be a system for finding information on a ground state of a photon using the quantum state in d-dimension.

The system 10 for calculating quantum chemistry based on a qudit may adjust parameters of a quantum circuit to find a ground state of a molecule. In other words, the system 10 for calculating quantum chemistry based on a qudit may be a system that is configured to perform a function such as a variational quantum eigensolver (VQE).

The quantum processing unit 100 is a device for storing and processing information using the characteristics of a quantum state provided in a quantum circuit. In this case, the quantum processing unit 100 may solve the quantum chemistry calculation using one or more optical components.

The classical processing unit 200 is a device for performing arithmetic and logical calculations using a processor and memory to process data. The classical processing unit 200 may solve mathematical calculations on the values measured by the quantum processing unit 100.

In this case, the quantum processing unit 100 and the classical processing unit 200 may be connected to transmit and receive output values processed by each. That is, the system 10 for calculating quantum chemistry based on a qudit may implement the variational quantum eigensolver using the quantum processing unit 100 and the classical processing unit 200 in conjunction.

With reference to FIG. 1, the quantum processing unit 100 includes a first update unit 110 and a second update unit 120.

The first update unit 110 updates a diffraction image. Specifically, the first update unit 110 updates the diffraction image on the basis of parameters obtained from the classical processing unit 200.

The first update unit 110 may generate a t-th diffraction image on the basis of an obtained t-th parameter, and update a previous diffraction image to a current diffraction image therefrom.

In this case, it is intended that the previous diffraction image is understood by those skilled in the art to be a (t−1)-th diffraction image by a (t−1)-th parameter generated by the same mechanism of the first update unit 110.

Meanwhile, it is of course understood that the first update unit 110 may update an (N−1)-th diffraction image to an N-th diffraction image on the basis of a subsequently obtained N-th parameter by the same mechanism.

In this case, N may be the number of times of updates performed by the quantum processing unit, which is determined by the classical processing unit described below.

That is, the first update unit 110 may update the diffraction image N times by sequentially obtaining first to N-th parameters. Ultimately, the first update unit 110 may generate the first to N-th diffraction images.

The first update unit 110 may update the diffraction image using a first spatial light modulator. Here, the diffraction image may be an image that gives a photon an orbital angular momentum state (OAM State).

The orbital angular momentum state is a state with angular momentum that exhibits spatial characteristics. The orbital angular momentum state has d modes that are orthogonal, and may be used as a basis for a qudit. That is, the photon may have a d-dimensional quantum state, by entering the diffracted image. In this case, d has a range of an infinite number of theoretically unlimited values.

The first update unit 110 is an optical component, which may generate the diffraction image using the first spatial light modulator. That is, the first spatial light modulator may generate the diffraction image on the basis of the values of the parameters determined by the classical processing unit 200. In this case, the parameters are attribute values for generating the diffraction image, which may include values corresponding to a diffraction grating, a phase, and intensity.

The second update unit 120 updates a state of the photon. Specifically, the second update unit 120 allow the photon to enter the generated diffraction image to update the state of the photon. The second update unit 120 may allow a photon in a 0-th state to enter the t-th diffraction image and be updated to a photon in a t-th state.

In this case, the 0-th state may mean an initial state of the photon before the photon is input to the quantum processing unit 100, and the t-th state may mean an updated state that the photon has after the photon entered the t-th diffraction image.

Meanwhile, the second update unit 120 may be designed on the basis of an optical component configured to cause the photon in the 0-th state to enter the diffraction image.

Meanwhile, the second update unit 120 may likewise allow the photon in the 0-th state to enter the first to N-th diffraction images generated by the first update unit 110, so that the second update unit 120 may collectively generate photons in the 0-th to N-th state.

That is, the quantum processing unit 100 may alternately execute the first and second update units 110 and 120 whenever the quantum processing unit 100 receives parameters sequentially from the classical processing unit 200. In this case, the quantum processing unit 100 may alternately update the first update unit 110 and the second update unit 120 N number of times, respectively. Here, t is a natural number greater than or equal to one and less than or equal to N, and N is preferably a natural number greater than or equal to one.

For example, the quantum processing unit 100 may, when N is two, allow the first update unit 110 to generate a first diffraction image on the basis of a first parameter, and then allow the second update unit 110 to diffract a photon in the 0-th state onto the first diffraction image to update the photon to a first state. Thereafter, the first update unit 110 again generates a second diffraction image on the basis of a second parameter, and then the second update unit 120 may diffract the photon in the 0-th state onto the second diffraction image to update the photon to a photon in a second state.

In contrast, when the quantum processing unit 100 receives no further parameter input from the classical processing unit 200, the quantum processing unit 100 may terminate execution of the first and second update units 110 and 120.

With reference back to FIG. 1, the quantum processing unit 100 may further include a measuring unit 130.

The measuring unit 130 may measure an expectation value of a Pauli operator corresponding to the photon in the t-th state.

In this case, the photon may be updated from the first to the N-th state, and the measuring unit 130 may measure expectation values of the Pauli operators corresponding to photons in the first state, the second state, . . . , the t-th state, and eventually the N-th state.

Specifically, the measuring unit 130 may measure the expectation value of the Pauli operator corresponding to the photon in the t-th state using a second spatial light modulator, which is an optical component.

The second spatial light modulator may be disposed to face the first spatial light modulator in parallel in a diagonal direction. The photon in the t-th state may propagate from the first spatial light modulator to the second spatial light modulator along a preset optical path. In this case, the optical path may be implemented by an optical component that includes a lens and an iris to prevent interference from the outside environment.

In this case, the measuring unit 130 may generate an inversion diffraction image through the second spatial light modulator to measure the expectation value of the Pauli operator corresponding to the photon in the t-th state.

In this case, the inversion diffraction image may be a diffraction image that converts a phase of the incident photon in the t-th state to a phase in the 0-th state. In other words, the inversion diffraction image may be an image that converts the phase of the photon in the t-th state to the phase in the 0-th state by an amount corresponding to a contribution of the Pauli operator.

The classical processing unit 200 includes a first calculation unit 210 and a determination unit 220.

The first calculation unit 210 and the determination unit 220 may be implemented by one or more processors provided in the classical processing unit 200, or by a combination of one or more processors, memory, and software, and may not be clearly distinguishable in specific operations in contrast to the examples illustrated.

The first calculation unit 210 calculates expectation values of Hamiltonians corresponding to the 0-th to N-th state. The first calculation unit 210 may calculate the expectation values of the Hamiltonians of the photons in the first to t-th state on the basis of the expectation values of the Pauli operators of the measuring unit 130.

The first calculation unit 210 may calculate an expectation value of the Hamiltonian corresponding to the photon in the t-th state by multiplying the expectation value of the Pauli operator corresponding to the photon in the t-th state by a preset weight coefficient.

The determination unit 220 determines the t-th parameter on the basis of an expectation value of the Hamiltonian corresponding to the (t−1)-th parameter.

The determination unit 220 may determine the t-th parameter such that the expectation value of the Hamiltonian corresponding to the t-th parameter is less than the expectation value of the Hamiltonian corresponding to the (t−1)-th parameter.

In one example, the determination unit 220 may determine an optimal parameter value for a diffraction image such that the expectation value of the Hamiltonian of the t-th parameter is less than the expectation value of the Hamiltonian of the (t−1)-th parameter as a value of the t-th parameter.

Here, the optimal parameter may mean a parameter such that the expectation value of the Hamiltonian of the t-th parameter is less than the expectation value of the Hamiltonian of the (t−1)-th parameter, but among which the parameter has the smallest value.

In this case, the determination unit 220 may determine the value of the t-th parameter on the basis of a COBYLA optimizer.

Meanwhile, the determination unit 220 may randomly determine a value of a parameter (first parameter) for generating an initial diffraction image (first diffraction image) as a preset value or a random value. That is, the first parameter may be determined independently of a 0-th parameter.

In another example, the determination unit 220 may randomly determine the value of the 0-th parameter that is the basis for the value of the parameter (first parameter) for generating the initial diffraction image (first diffraction image) as a preset value or as a random value.

The determination unit 220 may determine the parameters within a limited range. Specifically, the determination unit 220 may validly determine the value of the t-th parameter only when the value of the (t−1)-th parameter and the value of the t-th parameter are equal to or greater than the tolerance, to avoid unnecessary calculation repetitions.

For example, the determination unit 220 may determine the value of the t-th parameter as described above, but when a difference between the value of the t-th parameter and the value of the (t−1)-th parameter is equal to or less than a tolerance, the determination unit 220 may invalidate the value of the t-th parameter that was preliminarily determined.

In this case, the determination unit 220 may not determine any further parameters. The quantum processing unit 100 does not receive any further parameters, and may terminate the update.

In contrast, when the value of the t-th parameter is determined, but the difference between the value of the t-th parameter and the value of the (t−1)-th parameter is equal to or greater than the tolerance, the determination unit 220 may only definitively determine the value of the t-th parameter that was preliminarily determined.

In this case, the determination unit 220 inputs the t-th parameter to the quantum processing unit 100, and the quantum processing unit 100 may generate the photon in the t-th state according to the t-th parameter.

Meanwhile, the tolerance may preferably have a value of 0.01 or less.

The determination unit 220 may determine the parameters within a limited number of times. The determination unit 220 may determine the best parameter to be selected within a limited number of times of repetitions, considering when the ground state energy to be calculated does not converge.

Meanwhile, the parameter may be expressed in the form of a 2d-2 angular parameter. For example, when the orbital angular momentum state is four-dimensional, the parameter is preferably expressed numerically through six variables associated with the angles.

The classical processing unit 200 may further include a second calculation unit 230.

The second calculation unit 230 may calculate an eigenvalue of the Hamiltonian corresponding to the photon in the N-th state to calculate a ground state of the photon.

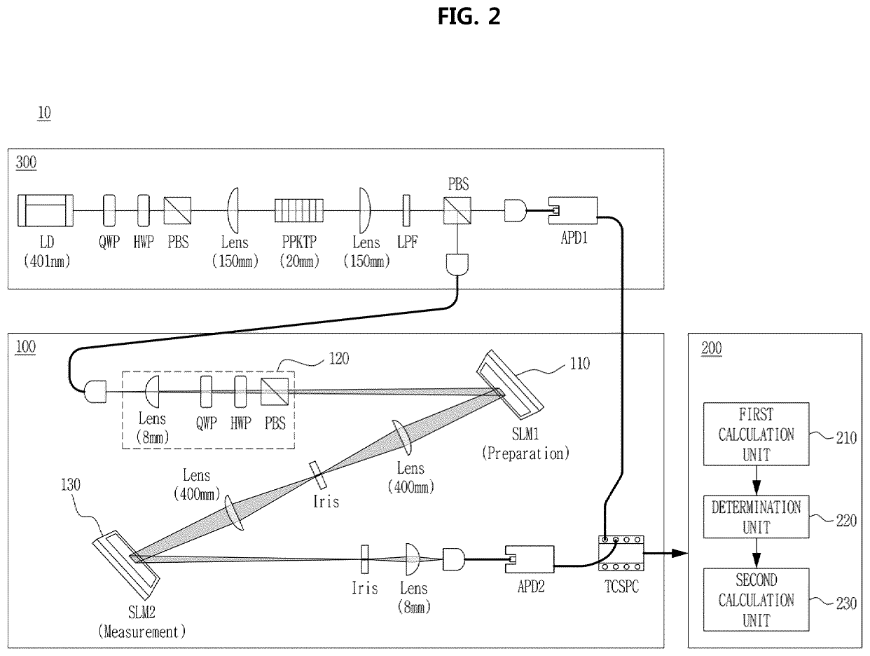

FIG. 2 is a view for describing a system for calculating quantum chemistry based on a qudit, according to an additional embodiment.

With reference to FIG. 2, the system 10 for calculating quantum chemistry based on a qudit may further include a photon generating device 300.

The photon generating device 300 may generate the photon on the basis of at least one of spontaneous parametric down conversion, a quantum dot, or a point defect.

For example, the photon generating device 300 may generate a pair of photons on the basis of the spontaneous parametric down conversion. The photon generating device 300 may separate the generated pair of photons on the basis of the spontaneous parametric down conversion to provide a single photon to be input to the quantum processing unit 100.

In this case, the photon generating device 300 may generate the single photon from a continuous wave using at least one of a light source, a polarization filter (e.g., a quarter-wave plate (QWP), a half-wave plate (HWP)), a frequency filter (e.g., a low pass filter (LPF), a high pass filter (HPF), a polarization beam splitter (PBS), a lens, or an optical crystal.

In a specific example, the photon generating device 300 may pump a continuous wave laser with a wavelength of 401 nm at a power of 47 mW onto a PPKTP-based crystal with a length of 20 mm, and generate a single photon on the basis of type-II spontaneous parametric down conversion.

The photon generating device 300 may generate the pair of photons by pumping the continuous wave laser onto the crystal. Then, the photon generating device 300 may transmit the single photon separated from the pair of photons to the quantum processing unit 100.

The photon generating device 300 may transmit the single photon in a first path, thereby causing the quantum processing unit 100 to solve a quantum chemistry problem through a single gate.

Meanwhile, the single photon is converted to a horizontally polarized state on the basis of an optical component provided in the first path, and the polarized single photon may enter the first spatial light modulator. For example, the optical component provided in the first path may include at least one of a lens, a polarizing filter, or a polarization beam splitter.

The photon generating device 300 may detect the other photon that is separated from the pair of photons. According to the detection of the other photon from the photon generating device 300, the quantum processing unit 100 may be assured that the single photon has been input. For example, the photon generating device 300 may detect the other photon using an avalanche photodiode (APD).

In another example, the photon generating device 300 may generate the single photon on the basis of a single photon source using at least one of a point defect or a quantum dot. In other words, the photon generating device 300 may provide the quantum processing unit 100 with the single photon itself, which is generated using at least one of a point defect or a quantum dot.

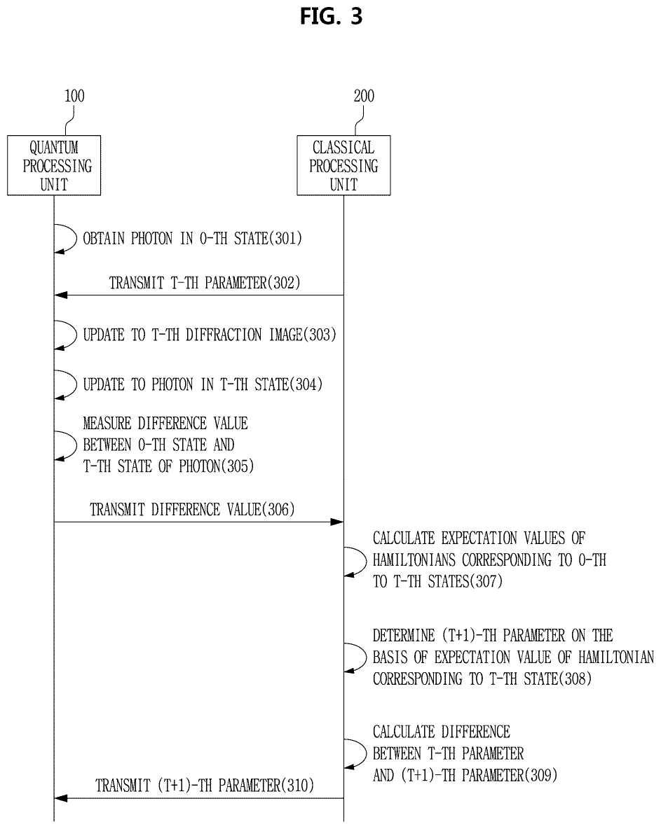

FIG. 3 is a flowchart for describing an operation sequence of one example performed by the system for calculating quantum chemistry based on a qudit according to one embodiment.

First, the quantum processing unit 100 obtains the photon in the 0-th state (301). Then, the classical processing unit 100 transmits the t-th parameter to the quantum processing unit 100 (302). That is, it may be understood that a t-th update of the quantum processing unit 100 is initialized.

Meanwhile, in FIG. 3, since continuous updating of the quantum processing unit 100 is assumed, it is assumed that t is a natural number equal to or greater than one, where t does not exceed N.

Then, the quantum processing unit 100 updates the diffraction image to the t-th diffraction image on the basis of the t-th parameter (303). Then, the quantum processing unit 100 allow the single photon to enter the t-th diffraction image to update the photon in the 0-th state to the t-th state (304). Then, the quantum processing unit 100 may measure the expectation value of the Pauli operator corresponding to the photon in the t-th state (305).
The quantum processing unit 100 may transmit the expectation value of the Pauli operator corresponding to the photon in the t-th state to the classical processing unit 200 (306). The classical processing unit 200 calculates 307 the expectation value of the Hamiltonian corresponding to the photon in the t-th state on the basis of the expectation value of the Pauli operator corresponding to the photon in the t-th state.

Then, the classical processing unit 200 determines a (t+1)-th parameter on the basis of the expectation value of the Hamiltonian corresponding to the photon in the t-th state (308).

In this case, the classical processing unit 200 may determine the (t+1)-th parameter that causes the expectation value of the Hamiltonian corresponding to a photon in the (t+1)-th state to be less than the expectation value of the Hamiltonian corresponding to the photon in the t-th state.

Thereafter, the classical processing unit 200 calculates a difference between the determined (t+1)-th parameter and the t-th parameter (309). For example, when a value of the difference exceeds the tolerance, the quantum processing unit 100 may perform t+1 updates using the (t+1)-th parameter for a precise inspection.

The classical processing unit 200 transmits the (t+1)-th parameter to the quantum processing unit 100 (310), and the quantum processing unit 100 and the classical processing unit 200 may repeat steps 303 to 310 again.

Meanwhile, it is obvious that a process for the photons in the first to (t−1)-th state may be likewise yielded by repeating steps 301 to 310.

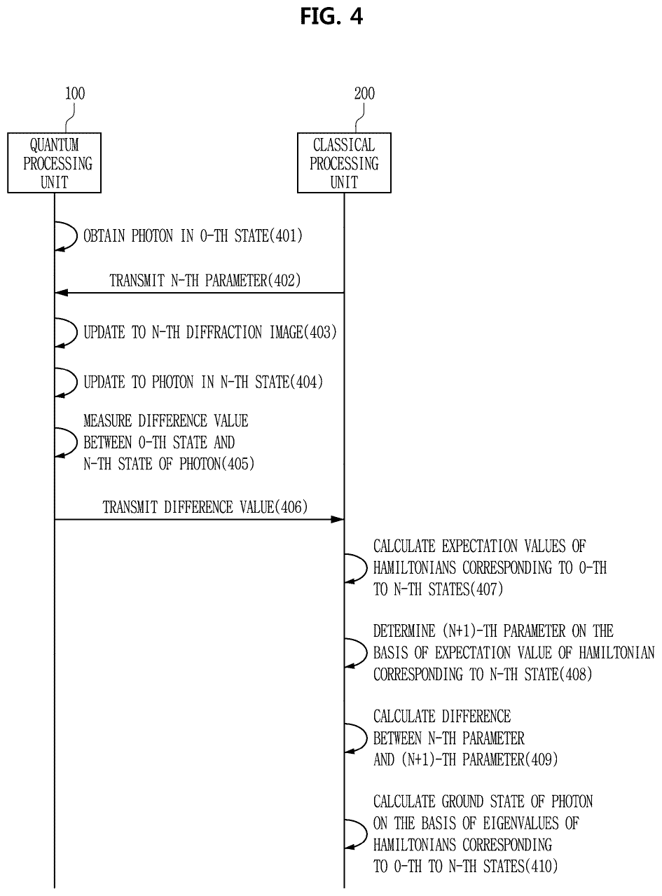

Hereinafter, FIG. 4 describes an operation sequence for performing a last (N-th) update of the system 10 for calculating quantum chemistry based on a qudit according to one embodiment when the value of the difference does not exceed the tolerance. FIG. 4 is a flowchart for describing an operation sequence of another example performed by the system for calculating quantum chemistry based on a qudit according to one embodiment.

Steps 401 to 409 in FIG. 4 are the same as steps 301 to 309 in FIG. 3, and the description is omitted where redundant.

When the difference between the preliminarily determined (N+1)-th parameter and the N-th parameter does not exceed the tolerance, the classical processing unit 200 does not transmit the (N+1)-th parameter to the quantum processing unit 100, and calculates the eigenvalue of the Hamiltonian corresponding to the N-th state. In this case, the classical processing unit 200 calculates a ground state of the photon on the basis of the eigenvalue of the Hamiltonian corresponding to the photon in the N-th state (410).

Meanwhile, the quantum processing unit 100 does not receive any further parameters, and each of the first update unit 110 and the second update unit 120 may perform only N updates and terminate the operation.

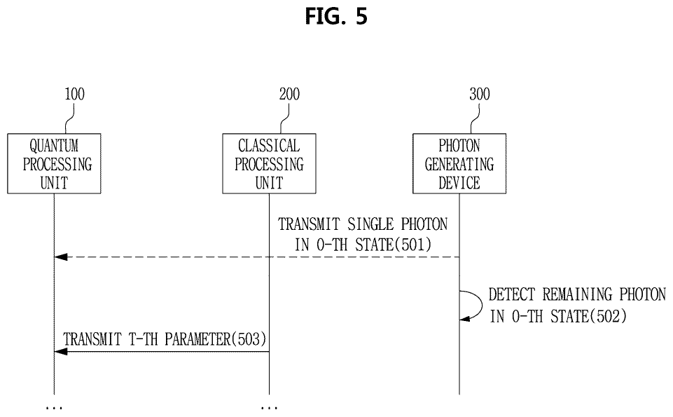

FIG. 5 is a flowchart for describing an operation sequence of one example performed by the system for calculating quantum chemistry based on a qudit according to the additional embodiment.

First, the photon generating device 300 transmits the single photon in the 0-th state to the quantum processing unit 100 (501). In this case, the single photon may be a single photon itself generated by the photon generating device 300, or may be one photon in the 0-th state separated from one pair of photons.

In this case, the single photon may be generated by the photon generating device 300 designed on the basis of at least one of a point defect or a quantum dot, and the pair of photons may be generated by the photon generating device 300 designed on the basis of the spontaneous parametric down conversion.

In particular, the photon generating device 300 based on the spontaneous parametric down conversion may detect the other photon that is separated from one pair of photons to identify whether the single photon has been input to the quantum processing unit 100 (502).

Here, when the photon generating device 300 does not detect the other photon, the photon generating device 300 may perform steps 501 and 502 again. In contrast, when the photon generating device 300 detects the other photon, the quantum processing unit 100 and the classical processing unit 200 may identically perform steps 302 to 310 and steps 402 to 410 described in FIGS. 3 and 4.

Meanwhile, step 502 is preferably omitted when the single photon generated by the photon generating device 300 is itself input to the quantum processing unit 100.

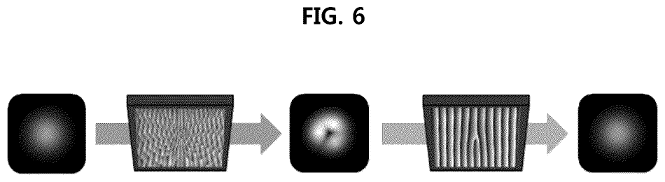

FIG. 6 is an exemplified view for describing a state of a photon being changed by the system for calculating quantum chemistry based on a qudit, according to one embodiment.

First, the quantum processing unit 100 receives the photon in the 0-th state as input by the photon generating device 300. In this case, the photon in the 0-th state has a first Gaussian mode. The first Gaussian mode is an initial state of a photon that has just been separated from a continuous wave, and may be defined, for example, as (1,p)=(0,0). In this case, 1 corresponds to an azimuth index expressed in integer units, and p corresponds to a radial index expressed in integer units.

Then, the quantum processing unit 100 may allow the photon in the 0-th state to enter the t-th diffraction image generated by the first spatial light modulator to be updated to the photon in the t-th state.

In this case, the photon in the t-th state may be converted to have a second Gaussian mode. Here, the second Gaussian mode may be a mode having an orbital angular momentum state. In other words, the second Gaussian mode may mean a qudit state that can be expressed as a vector in a d-dimensional Hilbert space that includes d orthogonal bases.

Then, the quantum processing unit 100 diffracts the photons in the t-th state onto the t-th inversion diffraction image generated by the second spatial light modulator. In this case, the inversion diffraction image may be an image generated such that when a photon having the t-th state is incident, the phase of the photon in the t-th state is converted to the phase of the 0-th state. In other words, the inversion diffraction image may be an image that converts the phase of the photon in the t-th state to the phase in the 0-th state by an amount corresponding to a contribution of the Pauli operator. The second spatial light modulator may measure the expectation value of the Pauli operator corresponding to the photon in the t-th state on the basis of the inversion diffraction image.

That is, the system 10 for calculating quantum chemistry based on a qudit according to one embodiment uses the diffraction image and the inversion diffraction image to measure the expectation value of the Pauli operator corresponding to the photon in the t-th state, which may replace the complex multi-qubit Pauli measurement originally used in the related art.

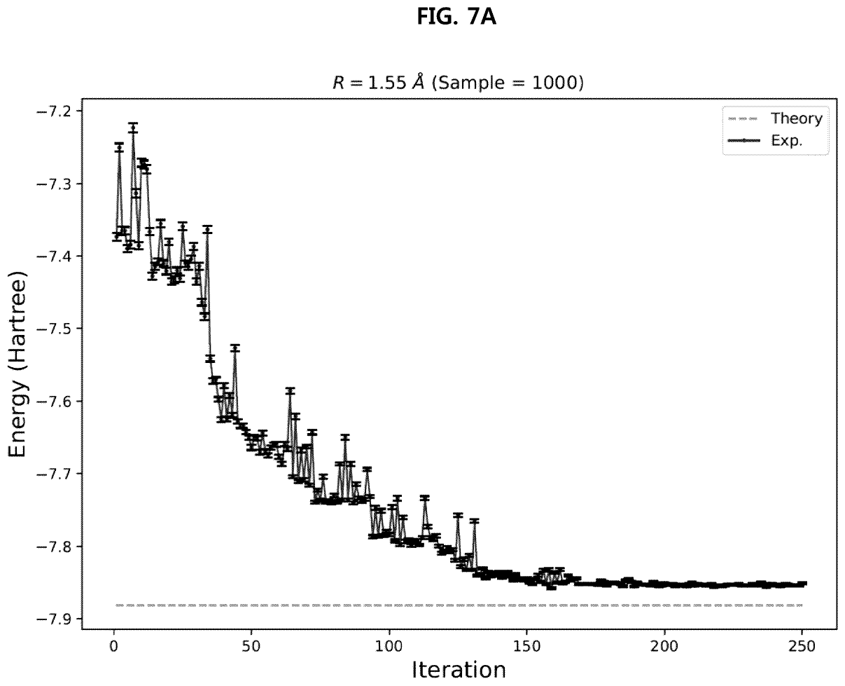

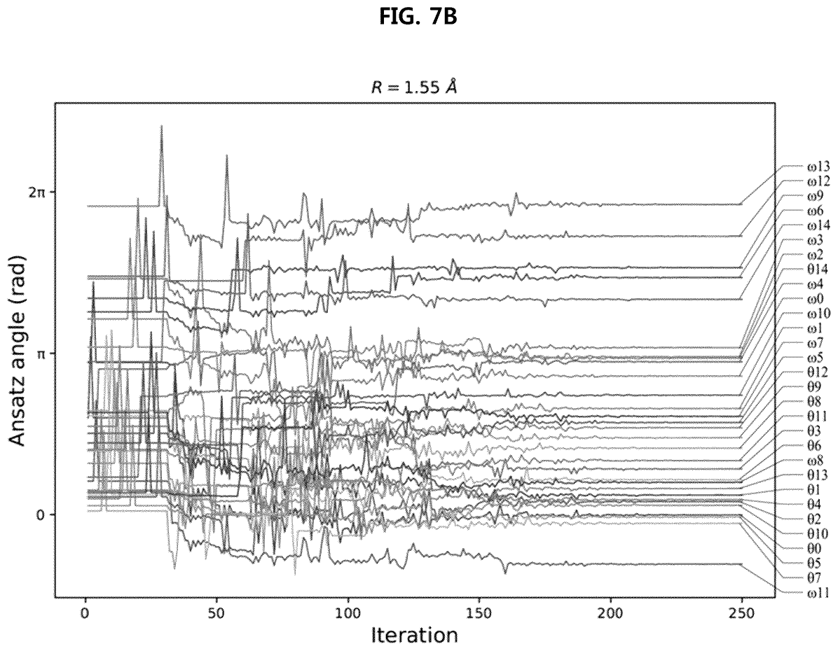

FIG. 7A and FIG. 7B are result graphs for describing a calculation efficiency of the system for calculating quantum chemistry based on a qudit according to one embodiment.

With reference to FIG. 7A, the ground state energy of a lithium hydride molecule derived by the system 10 for calculating quantum chemistry based on a qudit according to one embodiment is illustrated.

With reference to FIG. 7A, it can be seen that as the number of updates of the system 10 for calculating quantum chemistry based on a qudit according to one embodiment increases, the ground state energy converges to a specific value.

In this case, the system 10 for calculating quantum chemistry based on a qudit according to one embodiment may, to prevent excessive calculations, stop updating when the difference between the current parameter and the previous parameter is within the tolerance, and calculate the best value of the ground state during the performed update.

With reference to FIG. 7B, the parameters calculated by the system 10 for calculating quantum chemistry based on a qudit according to one embodiment are illustrated

With reference to FIG. 7B, it can be seen that as the number of updates of the system 10 for calculating quantum chemistry based on a qudit according to one embodiment increases, the value of the parameter converges to a specific value.

That is, the system 10 for calculating quantum chemistry based on a qudit according to one embodiment may stop updating when the difference between the current parameter and the previous parameter is within the tolerance, and calculate the best value of the ground state during the performed update.

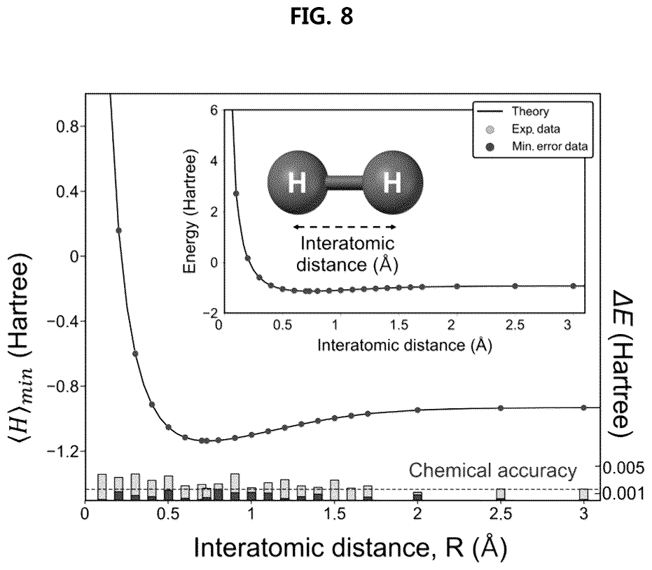

FIG. 8 is a result graph for describing a calculation precision of the system for calculating quantum chemistry based on a qudit according to one embodiment.

With reference to FIG. 8, the system 10 for calculating quantum chemistry based on a qudit according to one embodiment calculates the ground state energy based on the interatomic distance of a hydrogen molecule.

The qudit-based quantum chemistry calculation according to one embodiment solves the ground state energy according to the interatomic distance with high consistency with the theory, such that the average difference between the theoretical and experimental values reaches below chemical accuracy.

That is, the system 10 for calculating quantum chemistry based on a qudit according to one embodiment is verified for its high performance in achieving chemical accuracy, without the need for quantum error mitigation techniques to achieve chemical accuracy.

While the present invention has been described in detail above with reference to the representative exemplary embodiments, those skilled in the art to which the present invention pertains will understand that the exemplary embodiment may be variously modified without departing from the scope of the present invention. Therefore, the scope of the present invention should not be limited to the described exemplary embodiments, and should be defined by not only the claims to be described below, but also those equivalent to the claims.

### DESCRIPTION OF REFERENCE NUMERALS

10: System for calculating quantum chemistry

100: Quantum processing unit

110: First updating unit

120: Second updating unit

130: Measuring unit

200: Classical processing unit

210: First calculation unit

220: Determination unit

230: Second calculation unit

300: Photon generating device

What is claimed is:

1. A system for calculating quantum chemistry based on a qudit, the system comprising:
a quantum processing unit configured to include one or more optical-components; and
a classical processing unit configured to include one or more processors and memory,
wherein the quantum processing unit (hereinafter, N is a natural number equal to or greater than one, and t is a natural number equal to or greater than one and equal to or less than N) is configured to include:
a first updating unit, including one or more optical components, configured to update a diffraction image to a t-th diffraction image on the basis of a t-th parameter obtained from the classical processing unit; and
a second updating unit, including one or more optical components, configured to allow a photon in a 0-th state input to the quantum processing unit to enter the t-th diffraction image update to a photon in a t-th state,
wherein a first update unit and a second update unit are alternately updated N number of times sequentially from a first time to a N-th time, respectively, and
wherein the classical processing unit is configured to:
calculate expectation values of the first to t-th Hamiltonians corresponding to photons in first to t-th state, and
determine the t-th parameter on the basis of an expectation value of the Hamiltonian of a (t−1)-th parameter—a 0-th parameter being determined on the basis of an arbitrary value.

2. The system of claim 1, wherein the classical processing unit is further configured to calculate an eigenvalue of the Hamiltonian corresponding to a photon in an N-th state to calculate a ground state of the photon.

3. The system of claim 1, wherein the classical processing unit is configured to determine a value of the t-th parameter such that an expectation value of the Hamiltonian of the t-th parameter is less than the expectation value of the Hamiltonian of the (t−1)-th parameter.

4. The system of claim 1, wherein the classical processing unit is configured to determine a minimum value of the t-th parameter that causes an expectation value of the Hamiltonian of the t-th parameter to be less than the expectation value of the Hamiltonian of the (t−1)-th parameter.

5. The system of claim 1, wherein the classical processing unit is configured to determine a value of the t-th parameter when the difference between a value of the (t−1)-th parameter and the value of the t-th parameter is greater than or equal to a preset tolerance.

6. The system of claim 1, wherein the photon is single one.

7. The system of claim 1, further comprising:
a photon generating device configured to include one or more optical components,
wherein the photon generating device is configured to generate a single photon on the basis of at least one of spontaneous parametric down conversion, a point defect, or a quantum dot.

8. The system of claim 6, wherein the photon generating device is configured to generate a pair of photons on the basis of the spontaneous parametric down conversion, and
detect an other photon when a single photon separated from the pair of photons is input to the quantum processing unit.

9. The system of claim 5, wherein the quantum processing unit further include a measuring unit, including one or more optical components, configured to measure an expectation value of a Pauli operator corresponding to the photon in the t-th state, and
wherein the classical processing unit is configured to calculate an expectation value of the Hamiltonian of the photon in the t-th state on the basis of the expectation value of the Pauli operator corresponding to the photon in the t-th state.

10. The system of claim 1, wherein the first updating unit is configured to generate a diffraction image by a first spatial light modulator provided in the first update unit.

11. The system of claim 8, wherein the measuring unit is configured to measure an expectation value of a Pauli operator corresponding to a photon in the t-th state by a second spatial light modulator provided in the measuring unit.

12. The system of claim 1, wherein the second updating unit is configured to allow the photon to enter the diffraction image and update to have a qudit state in which the photon possesses an orbital angular momentum.
